(19) United States
(12) Patent Application Publication
(10) Pub. No.: US 2017/0119353 A1
(43) Pub. Date: May 4, 2017

(54) ULTRASOUND IMAGING SYSTEM TOUCHSCREEN USER INTERFACE

(71) Applicant: B-K Medical Aps, Herlev (DK)

(72) Inventors: Torben Svanberg Nielsen, Copenhagen (DK); Kaj Dunkan, Stenlille (DK); Jesper Helleso Hansen, Kobenhavn NV (DK); John Antol, Nahant, MA (US); Michael Leismann, Hvidovre (DK)

(73) Assignee: B-K Medical Aps, Herlev (DK)

(21) Appl. No.: 15/129,533

(22) PCT Filed: Mar. 27, 2014
(86) PCT No.: PCT/IB2014/060237

§ 371 Date: Sep. 27, 2016

(51) Int. Cl. A61B 8/00; G06F 3/041; G06F 3/0488

(52) U.S. Cl. CPC A61B 8/467; G06F 3/04883; G06F 3/0416; G06F 3/03549

(57) ABSTRACT

An ultrasound imaging system (102) includes a probe (104) with a transducer array (106) with at least one transducer element (108). The ultrasound imaging system further includes a console (112) with a controller (124) and an echo processor. The ultrasound imaging system further includes a display monitor (122). The ultrasound imaging system further includes a touch screen user interface (128), including: a touch panel (130) with a first major surface (402, 1402) and a first recess (408, 1408) in the first major surface; and at least one touch sensitive control (134) disposed in the recess. A method includes sensing a first physical contact with a touch control recessed in a surface of a touch screen user interface, generating a signal indicative of the gesture, sensing a second physical contact with the touch control recessed in the surface of the touch screen user interface, and performing a predetermined action based on the first and second physical contact.

### TECHNICAL FIELD

The following generally relates to an ultrasound imaging system and more particularly to an ultrasound imaging system touchscreen user interface.

### BACKGROUND

Ultrasound (US) imaging provides useful information about the interior characteristics (e.g., anatomical tissue, material flow, etc.) of a subject under examination. An ultrasound imaging system has included a probe with an ultrasound transducer array, a console, a display and a keyboard. The transducer array transmits an ultrasound signal into a field of view and receives echoes produced in response to the signal interacting with structure therein. The echoes are processed by the console, which generates images indicative of the structure that are visually presented in the display region.

An example suitable keyboard has a coherent, flat surface, without any holes, e.g. glass, combined with a touch screen and e.g. a TFT panel. Unfortunately, such a keyboard is not well-suited for navigation on the surface of the keyboard by the user's hand to locate and use a control of interest of the keyboard without the user having to look at the keyboard. Exacerbating the problem, some controls will have more than one function or mode. Furthermore, such a keyboard is not well-suited making measurements, etc. with high precision.

### SUMMARY

Aspects of the application address the above matters, and others.

In one aspect, an ultrasound imaging system includes a probe with a transducer array with at least one transducer element. The ultrasound imaging system further includes a console with a controller, which controls the at least one transducer element, and an echo processor. The ultrasound imaging system further includes a display monitor. The ultrasound imaging system further includes a touch screen user interface, including: a touch panel with a first major surface and a first recess in the first major surface; and at least one touch sensitive control disposed in the recess.

In another aspect, a method includes sensing a first physical contact with a touch control recessed in a surface of a touch screen user interface. The method further includes generating a signal indicative of the gesture. The method further includes sensing a second physical contact with the touch control recessed in the surface of the touch screen user interface. The method further includes performing a predetermined action based on the first and second physical contact.

In another aspect, an ultrasound imaging system including a console and a touch screen user interface. The console including transmit and receive circuitry, an echo processor, and a controller that controls the transmit circuitry and the receive circuitry and the echo processor. The touch screen user interface includes means for invoking a predetermined action of a touch control of the touch screen user interface.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example imaging system with a touchscreen control user interface;

FIG. 2 schematically illustrates an example touch panel of the touchscreen control user interface;

FIG. 3 schematically illustrates a top down view of an example trackball touch control of an active region of the panel of the touchscreen control user interface;

FIG. 4 schematically illustrates a cross-sectional view of the example trackball touch control of FIG. 3;

FIG. 5 schematically illustrates a perspective view of the example trackball touch control of FIG. 3;

FIGS. 6-9 illustrates example operation of the example trackball touch control of FIG. 4;

FIGS. 10-13 illustrates variations of the example trackball touch control of FIGS. 4 and 14;

FIG. 14 schematically illustrates a top down view of an example toggle touch control of the active region of the panel of the touchscreen control user interface;

FIG. 15 schematically illustrates a cross-sectional view of the example toggle touch control of FIG. 14;

FIG. 16 schematically illustrates a perspective view of the example toggle touch control of FIG. 14;

FIGS. 17-20 illustrates example operation of the example toggle touch control of FIG. 14; and

FIG. 21 illustrates an example method in accordance with the embodiments disclosed herein.

### DETAILED DESCRIPTION

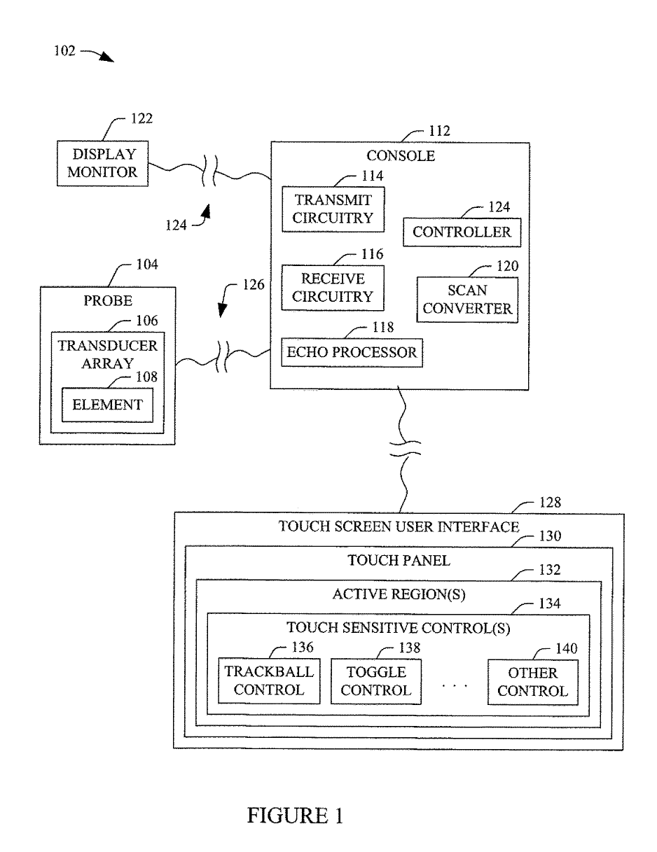

FIG. 1 schematically illustrates an ultrasound (US) imaging system 102. The ultrasound imaging system 102 includes a probe 104 with a one-dimensional (1D) or two-dimensional (2D) transducer array 106 with at least one transducer element 108. The at least one transducer element 108 is configured to transmit ultrasound signals and receive echo signals. Suitable array configurations include, but are not limited to, linear, curved (e.g., concave, convex, etc.), circular, etc., full populated or sparse, etc.

The ultrasound imaging system 102 further includes a console 112. The console 112 includes transmit circuitry 114 that selectively excites one or more of the at least one transducer element 108. More particularly, the transmit circuitry 114 generates a set of pulses (or a pulsed signal) that are conveyed to the transducer array 106. The set of pulses excites the at least one transducer element 108, causing the at least one transducer element 108 to transmit an ultrasound signal into an examination scan field of view.

The console 112 further includes receive circuitry 116 that receives a set of echoes (or echo signals) generated in response to the transmitted ultrasound signals. The echoes, generally, are a result of the interaction between the emitted ultrasound signals and the object (e.g., flowing blood cells, organ cells, etc.) in the scan field of view. The receive circuit 116 may be configured for spatial compounding, filtering (e.g., FIR and/or IIR), and/or other echo processing.

The console 112 further includes an echo processor (e.g., a beamformer) 118 that processes the received echoes. For example, in B-mode, this may include applying time delays and weights to the echoes and summing the delayed and weighted echoes, and generating an image. The console 112 further includes a scan converter 120 that scan converts the processed data for display, e.g., by converting the beamformed data to the coordinate system of a display monitor used to visually present the processed data.

The console 112 further includes a controller 124 that controls the various components of the system 102. For example, such control may include controlling the transmit circuitry 114 to excite individual or groups of the at least one transducer element 108 for an A-mode, B-mode, C-plane, and/or other data acquisition mode, steering and/or focusing the transmitted signal, etc., actuating the at least one transducer element 108 for steering and/or focusing the received echoes, etc.

The ultrasound imaging system 102 further includes a display monitor 122. The display monitor 122 can be a cathode ray tube (CRT), a liquid crystal display (LCD), a light emitting diode (LED), and/or other display monitor. The display monitor 122 includes a display region, which can visually present images and/or flow information generated by the console 112.

The system 102 further includes a touch screen user interface 128 with a touch panel 130. The touch panel 130 includes a resistive, a capacitive, an acoustic, an infrared, an optical, a piezoelectric, and/or other region. The touch panel 130 includes an active region(s) 132 with a touch sensitive control(s) 134. A touch sensitive control(s) 134 is actuated by a gesture (e.g., a press, a swipe, a touch, etc.) on the touch sensitive control(s) 134 with one or more fingers, a stylus, a glove, etc.

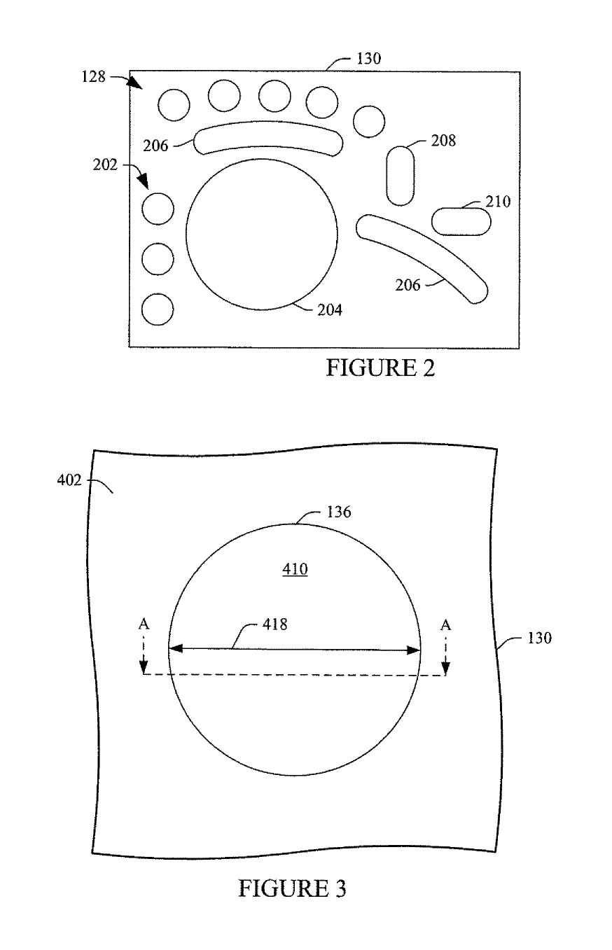

Examples of the touch sensitive control(s) 134 include a trackball control 136 for navigating a graphical pointer displayed in the display region of the display monitor 122, a toggle control 138 for incrementing and decrementing value corresponding to focus, depth, zoom, etc. and/or other control(s) 140. Briefly turning to FIG. 2, an example of the touch screen user interface 128 with a plurality of the touch sensitive control(s) 134 is illustrated.

In FIG. 2, the touch sensitive control(s) 134 include circular shaped controls 202 and 204. The controls 202 have a first size (i.e., diameter), and the control 204 has a second size, which is larger than the first size. In a variation, the touch sensitive control(s) 134 include only a single size circular control or more than two sizes of circular controls. Furthermore, the illustrated number of the circular shaped controls 202 and 204 is not limiting; in a variation, there could be more or less of the circular controls 202 and 204.

The touch sensitive control(s) 134 also include rectangular shaped controls 206 in which the rectangular shaped controls 206 include curved sides and rounded corners. In this example, the rectangular shaped controls 206 are the same size. However, in a variation, the touch sensitive control(s) 134 include multiple different size rectangular shaped controls 206. Furthermore, the illustrated number of the rectangular shaped controls 206 is not limiting; in a variation, there could be more or less of the circular controls 202 and 204.

The touch sensitive control(s) 134 also include oval or elliptical shaped controls 208 and 210. In this example, the elliptical shaped controls 208 and 210 are the same size. However, in a variation, the touch sensitive control(s) 134 include multiple different size elliptical shaped controls 208 and 210. Furthermore, the illustrated number of the elliptical shaped controls 208 and 210 is not limiting; in a variation, there could be more or less of the elliptical shaped controls 208 and 210.

Returning to FIG. 1, the touch panel 130 may also include one or more non-active regions, a display such as a LCD, a thin film transistor (TFT) LCD, an organic light-emitting diode (OLED) and/or other display, and/or other features. Another example of a suitable interface is described in U.S. patent application Ser. No. 13/748,653, which was filed on Jan. 24, 2013, and entitled “Ultrasound Imaging System,” which is incorporated herein by reference in its entirety.

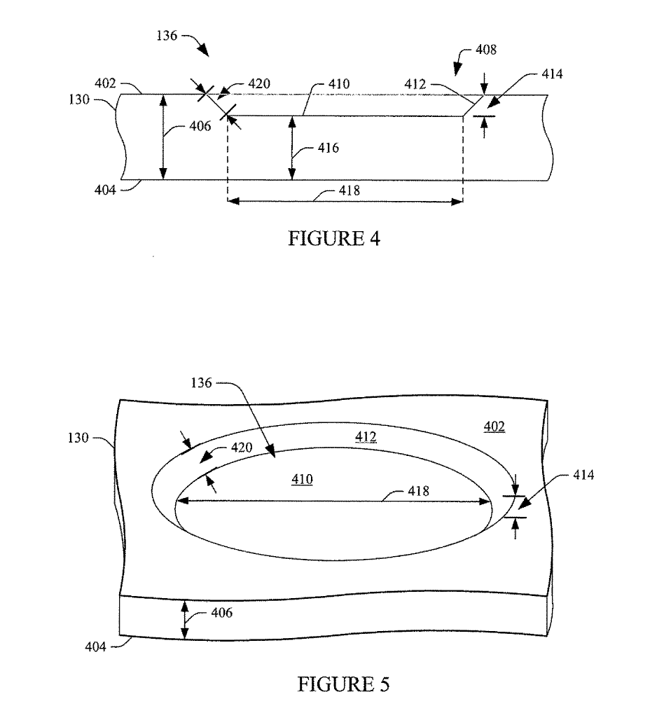

FIGS. 3, 4 and 5 illustrate an example of the trackball control 136 (FIG. 1) implemented using the larger circular shaped control 204 (FIG. 2). FIG. 3 shows a top down view of the trackball control 136 in connection with a sub-portion of the touch panel 130. FIG. 4 shows a cross-sectional view of the trackball control 136 along lines A-A. FIG. 5 shows a perspective view of the trackball control 136.

With reference to FIGS. 3, 4 and 5, the trackball control 136 has a first major surface 402 and a second major surface 404, which is parallel to and opposite from the first major surface 402. The first and second major surfaces 402 and 404 are separated by a material of the touch panel 130. In the illustrated embodiment, the first and second major surfaces 402 and 404 are separated are separated by a non-zero distance 406, which is in a range of 1 to 6 millimeters (mm).

The first major surface 402 includes a recess 408. The recess has a generally flat, planar active surface 410 and a side wall 412, which extends from the first major surface 402 to the generally flat, planar active surface 410 within the touch panel 130. The generally flat, planar surface 410 is offset from the first major surface 402 by a non-zero distance 414, which is in a range of 0.1 to 1 mm.

The recess 408 has a diameter 418, which is in a range of 40 to 100 mm. The illustrated side wall 412 extends linearly or non-linearly from the first major surface 402 to the recess 408. The side wall 412 has a length 420, which is in a range of 0.1 to 4 mm. It is to be appreciated that the distances 406, 414, 416, 418, and 420 are provided for explanatory purposes and are not limiting. Other ranges for these distances are contemplated herein.

The circular trackball control 136 in FIG. 3-5 resembles a trackball and thereby is easily recognized by ultrasound users. Configured to provide trackball functionality, the circular trackball control 136 can be used to control a cursor on the ultrasound image with higher accuracy compared to managing the ultrasound image with direct touch input. Using the circular trackball control 136 as such, the circular trackball control 136 can be used to make measurements, etc. with high precision.

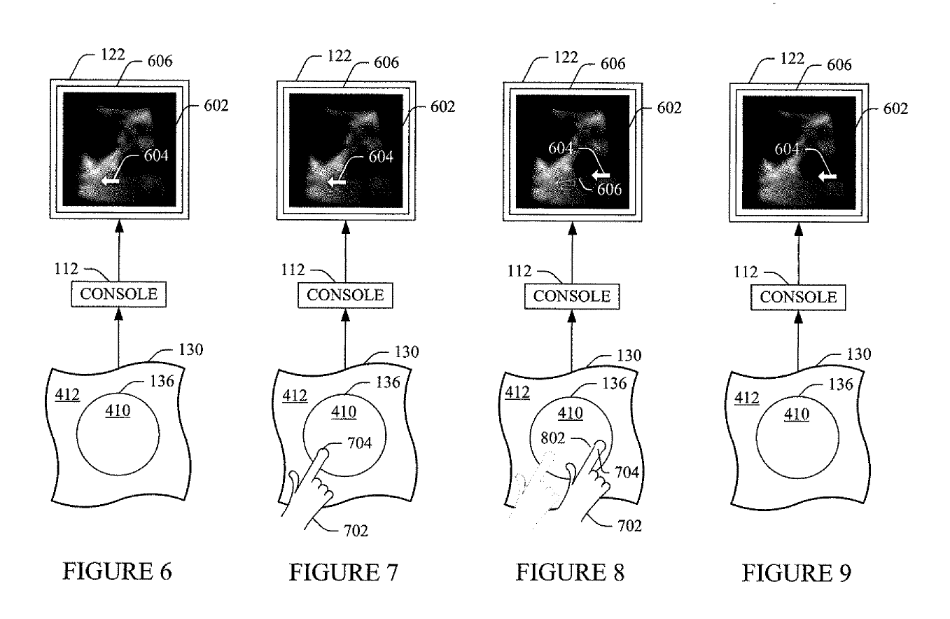

FIGS. 6, 7, 8 and 9 show example operation of the trackball control 136 of FIGS. 3, 4 and 5. FIG. 6 shows the sub-portion of the touch panel 130 with the trackball control 136 in connection with the console 112 and the display 122, which displays an image 602 and displays a graphical cursor 604 (e.g., an arrow in the illustrated embodiment) in a display region 606 of the display 122.

In FIG. 7, a user 702 touches, with a finger 704, the generally flat, planar active surface 410. In response thereto, the touch screen user interface 128 conveys a signal to the console 112. The signal indicates a current location of the touch on the generally flat, planar active surface 410 of the trackball control 136. The controller maps the current finger position to a current location of the graphical cursor 604 on the image 602.

In FIG. 8, the user 702 slides the finger 704 across the generally flat, planar active surface 410. As the finger slides, the touch screen user interface 128 conveys a signal to the console 112. The signal indicates each new current location of the touch on the generally flat, planar active surface 410 of the trackball control 136. The controller 124 moves the graphical cursor 604 along a path 606 that corresponds to the movement of the finger cross the generally flat, planar active surface 410.

In FIG. 9, the user 702 removes the finger 704 from the generally flat, planar active surface 410. FIG. 9 shows the sub-portion of the touch panel 130 with the trackball control 136 in connection with the console 112 and the display 122, which displays the image 602 and the graphical cursor 604, at the new location, in the display region 606 of the display 122.

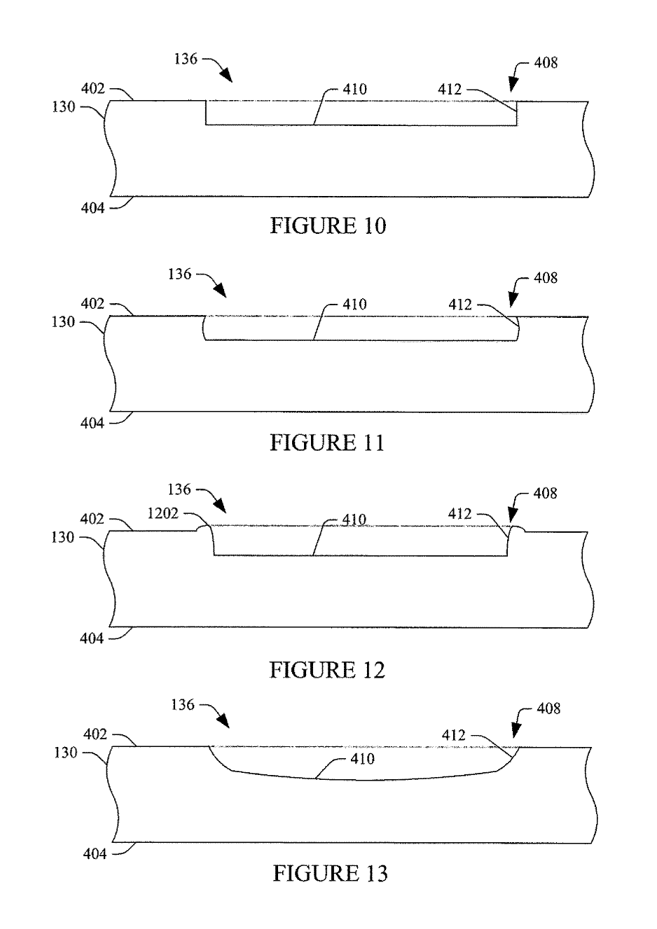

FIGS. 10, 11, 12 and 13 show variations of the trackball control 136 of FIGS. 3, 4 and 5. In FIG. 10, the side wall 412 extends generally perpendicular between the first major surface 402 and the generally flat, planar active surface 410. In FIG. 10, the side wall 412 is a curved surface. In FIG. 12, the first major surface 402 and the side wall 412 meet at a region 1202 that is raised above first major surface 402. In FIG. 13, the side wall 412 is part of the first major surface 402, which includes a curved active region.

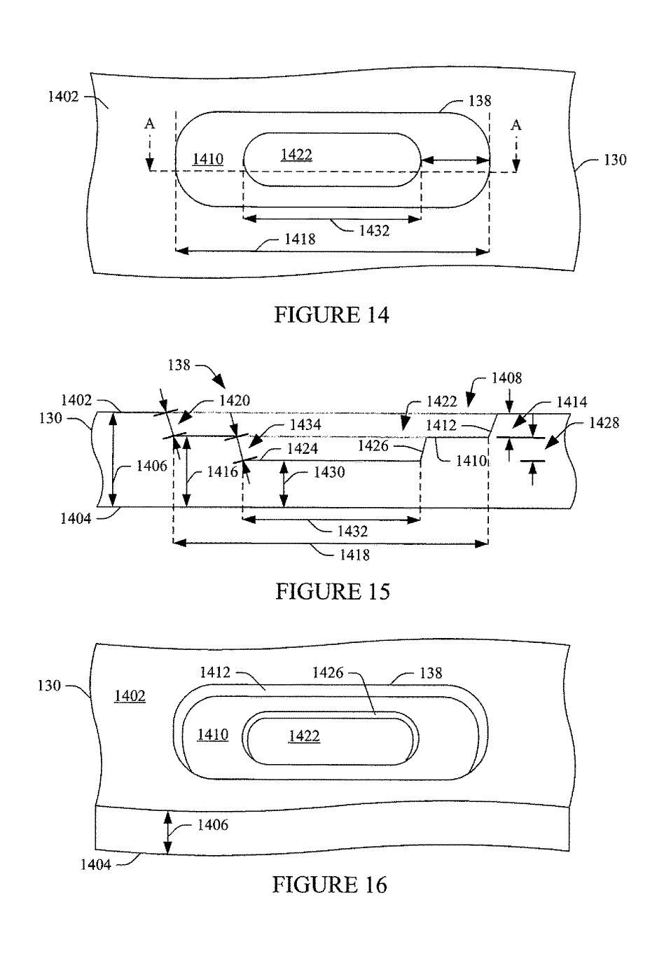

FIGS. 14, 15 and 16 illustrate an example of the toggle control 138 (FIG. 1) implemented using the elliptical shaped control 208 and 210 (FIG. 2). FIG. 14 shows a top down view of the toggle control 138 in connection with a sub-portion of the touch panel 130. FIG. 15 shows a cross-sectional view of the toggle control 138 along lines A-A. FIG. 16 shows a perspective view of the toggle control 138.

The toggle control 138 has a first major surface 1402 and a second major surface 1404, which is parallel to and opposite from the first major surface 1402. The first and second major surfaces 1402 and 1404 are separated by a material of the touch panel 130. In the illustrated embodiment, the first and second major surfaces 1402 and 1404 are separated are separated by a first non-zero distance 1406, which is in a range of 1 to 6 mm.

The first major surface 1402 includes a first elliptical recess 1408. The first elliptical recess 1408 has a generally flat, planar active surface 1410 and a first side wall 1412, which extends from the first major surface 1402 to the generally flat, planar active surface 1410 within the touch panel 130. The generally flat, planar surface 1410 is offset from the first major surface 1402 by a second non-zero distance 1414, which is in a range of 0.1 to 0.5 mm and from the second major surface 1404 by a first non-zero distance 1416.

The first elliptical recess 1408 has a first long axis 1418, which is in a range of 20 to 40 mm. The illustrated first side wall 1412 extends linearly from the first major surface 1402 to the first generally flat, planar surface 1410. The first side wall 1412 has a first length 1420, which is in a range of 0.1 to 2 mm. It is to be appreciated that the distances 1406, 1414, 1416, 1418, and 1420 are provided for explanatory purposes and are not limiting. Other ranges for these distances are contemplated herein.

The first elliptical recess 1408 includes a second elliptical recess 1422. The second elliptical recess 1422 has a generally flat, planar actuating surface 1424 and a second side wall 1426, which extends from the generally flat, planar surface 1410 to the generally flat, planar actuating surface 1424 within the touch panel 130. The generally flat, planar actuating surface 1424 is offset from the generally flat, planar active surface 1410 by a third non-zero distance 1428, which is in a range of 0.1 to 0.5 mm, and from the second major surface 1404 by a fourth non-zero distance 1430.

The second elliptical recess 1422 has a second long axis 1432, which is in a range of 2 to 30 mm. The illustrated second side wall 1426 extends linearly or non-linearly from the generally flat, planar active surface 1410 to the generally flat, planar actuating surface 1424. The second side wall 1426 has a second length 1434, which is in a range of 0.1 to 2 mm. It is to be appreciated that the distances 1428, 1430, 1432 and 1434, are provided for explanatory purposes and are not limiting. Other ranges for these distances are contemplated herein.

In a variation, at least one of the first or the second recesses 1408 or 1422 is circular, rectangular, square, and/or otherwise shaped.

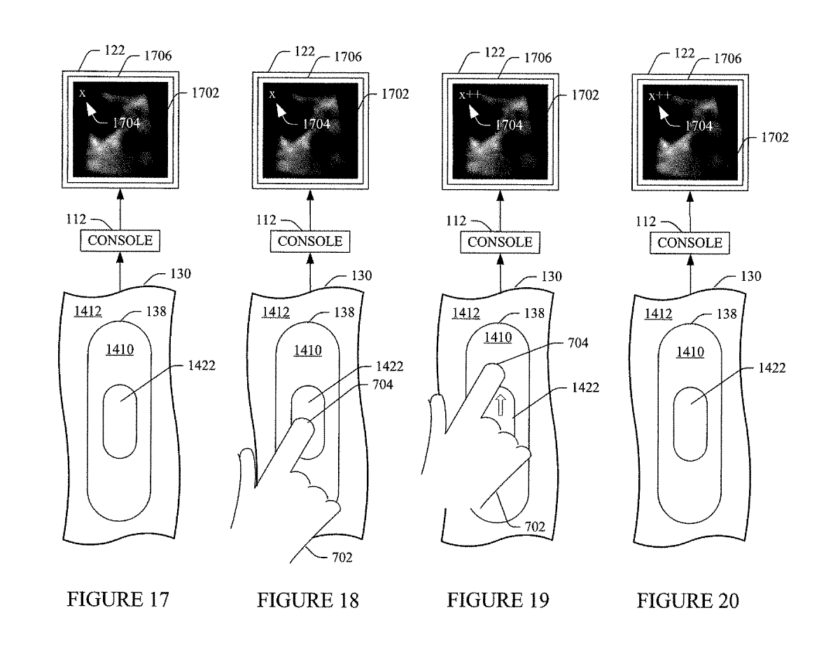

FIGS. 17, 18, 19 and 20 show example operation of the toggle control 138 of FIGS. 14, 15 and 16. FIG. 17 shows the sub-portion of the touch panel 130 with the toggle control 138 in connection with the console 112 and the display 122, which displays an image 1702 and a graphical indicia 1704 representing a numerical value in a display region 1706 of the display 122.

In FIG. 18, the user 702 touches, with the finger 704, the generally flat, planar actuating surface 1422. In response thereto, the touch screen user interface 128 generates a control activation signal, which activates the generally flat, planar active surface 1410.

In FIG. 19, the user 702 slides the finger 704 from the generally flat, planar actuating surface 1422 to the generally flat, planar active surface 1410. In response to the finger 704 being on the generally flat, planar active surface 1410, the controller increments the value 1704. In one instance, the controller increments the value 1704 again after a predetermined time delay from the previous increment where the finger 704 remains on the generally flat, planar active surface 1410.

In another instance, the controller increments the value 1704 again after a predetermined time delay from the previous increment in response to the user 702 removing the finger 704 and then touching the generally flat, planar active surface 1410 again. In another instance, a combination and/or other gesture is used to increment the value 1704 again. In the illustrated embodiment, the value 1704 is incremented twice as indicated by the two pluses (“++”).

In FIG. 20, the user 702 removes the finger 704 from the toggle control 138. In response to a predetermined time delay from the removal of the finger 704, the controller deactivates the generally flat, planar actuating surface 1422.

To decrement the value 1704, the user 702 performs the above, but in the opposite direction as that shown in the FIGS. 17-20. That is, in this embodiment, moving the finger 704 up the long axis increments the value 1704 and moving the finger 704 down the long axis decrements the value 1704. In a variation, moving the finger 704 up the long axis decrements the value 1704 and moving the finger 704 down the long axis increments the value 1704.

In the illustrated embodiment, moving the finger 704 perpendicular to the long axis does not change the value 1704. In a variation, moving the finger 704 as described above increments and decrements the value 1704 in accordance with a first predetermined value (e.g., 1), and moving the finger 704 perpendicular to the long axis increments and decrements the value 1704 in accordance with a second different predetermined value (e.g., 5).

Similar to the trackball control 136, the transition between the first major surface 1402 to the generally flat, planar active surface 410 and/or between the generally flat, planar active surface 410 and the generally flat, planar actuating surface 422 can be as shown in FIGS. 10, 11, 12 and 13 and/or otherwise.

Generally, the elliptical toggle control 138 of FIG. 14-16 includes a double indentation, which provides a haptic input, thereby helping a user operate the elliptical toggle control 138 without looking at the touch panel 130. The lower level of the indentation is used to activate the upper level, which is used to manipulate the control. Where the elliptical toggle control 138 is configured as a focus bar, the lower level is used to activate the upper level and to identify whether to increase or decrease the focus bar, and the upper level increases or decreases the size in response to the appropriate gesture.

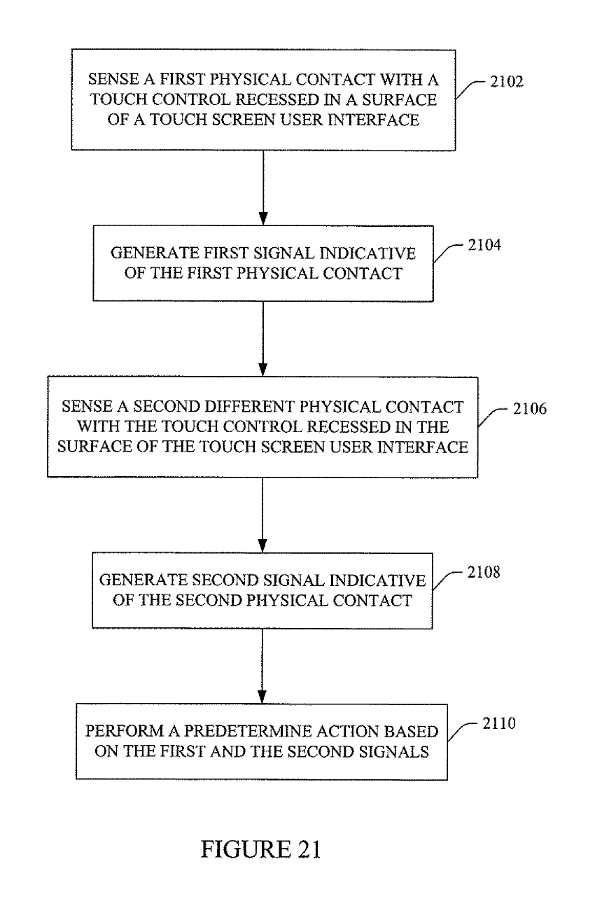

FIG. 21 illustrates a method in accordance with the embodiments disclosed herein.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 2102, a first physical contact with a touch control recessed in a surface of a touch screen user interface is sensed.

At 2104, a first signal indicative of the first physical contact is generated.

At 2106, a second different physical contact with the touch control recessed in the surface of the touch screen user interface is sensed; and

At 2108, a second signal indicative of the second physical contact is generated.

At 2110, a predetermined action is performed based on the first and second physical contact.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An ultrasound imaging system, comprising:
a probe, including: a transducer array with at least one transducer element;
a console, including: a controller that controls the at least one transducer element; and an echo processor;
a display monitor; and
a touch screen user interface, including: a touch panel with a first major surface and a first recess in the first major surface; and at least one touch sensitive control disposed in the recess.

2. The ultrasound imaging system of claim 1, wherein the at least one touch sensitive control is circular in shape.

3. The ultrasound imaging system of claim 2, wherein the at least one touch sensitive control has a diameter in a range of 40 to 100 mm and a depth in a range of 0.1 to 1 mm.

4. The ultrasound imaging system of claim 2, the first recess, comprising:
a touch sensitive flat, planar surface that senses a first gesture thereon which activates the touch sensitive flat, planar surface, and that senses a subsequent gesture thereon that invokes a predetermined action of the at least one touch sensitive control.

5. The ultrasound imaging system of claim 1, wherein the at least one touch sensitive control is touch sensitive trackball, and wherein the predetermined move causes the control to move a graphical pointer displayed via the display in coordination with the gesture.

6. The ultrasound imaging system of claim 1, wherein the at least one touch sensitive control is elliptical in shape.

7. The ultrasound imaging system of claim 6, wherein the first recess has a first long axis in a range of 20 to 40 mm and a first depth in a range of 0.1 to 0.5 mm.

8. The ultrasound imaging system of claim 6, the first recess, comprising:
a second recess disposed therein.

9. The ultrasound imaging system of claim 8, wherein the second recess is elliptical in shape.

10. The ultrasound imaging system of claim 8, wherein the second recess has a second long axis in a range of 20 to 30 mm and a second depth in a range of 0.1 to 0.5 mm.

11. The ultrasound imaging system of claim 6, the first recess, comprising:
a touch sensitive flat, actuating planar surface; and
the second recess, comprising:
a touch sensitive flat, active planar surface,
wherein the touch sensitive flat, actuating planar surface senses a first gesture thereon, which activates the touch sensitive flat, active planar surface.

12. The ultrasound imaging system of claim 11, wherein moving the first gesture from the touch sensitive flat, actuating planar surface to the touch sensitive flat, active planar surface invokes a predetermined action of the at least one touch sensitive control.

13. The ultrasound imaging system of claim 12, wherein the at least one touch sensitive control is toggle control, and wherein moving the first gesture in one direction invokes from the touch sensitive flat, actuating planar surface to the touch sensitive flat, active planar surface increments a control parameter and moving the first gesture in an opposite direction from the touch sensitive flat, actuating planar surface to the touch sensitive flat, active planar surface decrements the control parameter.

14. A method, comprising:
sensing a first physical contact with a touch control recessed in a surface of a touch screen user interface;
generating a signal indicative of the gesture;
sensing a second physical contact with the touch control recessed in the surface of the touch screen user interface; and
performing a predetermined action based on the first and second physical contact.

15. The method of claim 14, wherein the touch control is circular in shape, and further comprising:
moving graphical indicia, which is displayed on a display monitor, in coordination with the second physical contact.

16. The method of claim 15, wherein the second physical contact is on a same area of the touch control as the first physical contact.

17. The method of claim 16, wherein the touch control is elliptical in shape, and further comprising:
at least one of incrementing or decrementing a numerical value in response to the second physical contact.

18. The method of claim 17, wherein the first physical contact is in a first recess of the touch control.

19. The method of claim 18, wherein the second physical contact is in a second recess of the touch control, wherein the first recess is a recess inside of the second recess.

20. An ultrasound imaging system, comprising:
a console, including:
transmit and receive circuitry;
an echo processor; and
a controller that controls the transmit and receive circuitry and the echo processor; and
a touch screen user interface, including:
means for invoking a predetermined action of a touch control of the touch screen user interface.
